(19) United States
(12) Patent Application Publication
(10) Pub. No.: US 2007/0026014 A1
(43) Pub. Date: Feb. 1, 2007

(54) Interferon beta in severe acute respiratory syndrome (sars)

(75) Inventor: Giampiero de Luca, Conches-Geneva (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ARES TRADING S.A., AUBONNE (CH)

(21) Appl. No.: 10/553,135

(22) PCT Filed: Apr. 6, 2004
(86) PCT No.: PCT/EP04/50456

§ 371 Date: Jun. 29, 2006

(30) Foreign Application Priority Data
Apr. 17, 2003 (EP) 03101066.3

(51) Int. Cl. A61K 39/395; A61K 39/12

(52) U.S. Cl. 424/204.1; 424/130.1

(57) ABSTRACT

The us of an interferon (IFN) for the manufacture of a medicament useful for treatment and/or prevention of Severe Acute Respiratory Syndrome (SARS) is described in the present invention.

### FIELD OF THE INVENTION

The present invention relates to the use of an interferon (IFN) for the manufacture of a medicament for treatment and/or prevention of Severe Acute Respiratory Syndrome (SARS)

### BACKGROUND OF THE INVENTION

Pneumonia (pneumonitis) in an acute infection of lung parenchyma including alveotar spaces and interstitial tissue. It may affect an entire lobe (lobar pneumonia), a segment of a lobe (segmental or lobular pneumonia), alveoil contiguous to bronchi (bronchopneumonia), or interstitial tissue (interstitial pneumonia). These distinctions are generally based on x-ray observations.

Bacteria are the most common cause of pneumonia in adults >30 yr. Of these, Streptococcus pneumoniae is the most common. Other pathogens include anaerobic bacteria, Staphylococcus aureus, Haemophilus influenzae, Chlamydia pneumoniae, C. psittaci, C. trachomatis, Moraxella (Branhamella) catarrhalis, Legionella pneumophila, Klebsiella pneumoniae, and other gram-negative bacilli. Mycoplasma pneumoniae, a bacteria-like organism, is particularly common in older children and young adults, typically in the spring. Major pulmonary pathogens in infants and children are viruses: respiratory syncytial virus, parainfluenza virus, and influenza A and B viruses. These agents may also cause pneumonia in adults; however, the only common viruses in previously healthy adults are influenza A, occasionally influenza B, and rarely variclla-zoster. Among other agents are higher bacteria including Nocardia and Actinomyces sp; mycobacteria, including Mycobacterium tuberculosis and atypical strains (primarily M. kansasii and M. avium-intracellulare); fungi, including Histoplasma capsulatum, Coccidioides immitis, Blastomyces dermatitdis, Cryptococcus neoformans, Aspergillus fumigates, and Pneumocystis carinii; and rickettsiae, primarily Coxiella bumetii (Q fever).

Typical symptoms include cough, fever, and sputum production, usually developing over days and sometimes accompanied by pleurisy. Physical examination may detect tachypnea and signs of consolidation, such as crackles with bronchial breath sounds. This syndrome is commonly caused by bacteria, such as S. pneumoniae and H. Influenzae.

Diagnosis is based on the characteristic symptoms combined with an infiltrate on chest x-ray.

About 30 to 50% of patients have no identifiable pathogen despite a clinical impression of bacterial pneumonia Although the time-honored method of identifying bacterial pathogens is culturing expectorated sputum, these specimens are often misleading because normal oropharyngeal flora may contaminate them during passage through the upper airways. The most reliable specimens are taken from normally sterile sites, such as blood in patients with bacteremic pneumonia or pleural fluid in patients with empyema. Special culture techniques, special stains, serologic assays, or lung biopsies are required to identify some pathogens: mycobacteria, mycoplasmas, anaerobic bacteria, chlamydiae, viruses, fungi, legionellae, rickettsiae, and parasites.

Treatment consists of respiratory support, including O₂ if indicated, and antibiotics, which are selected on the basis of Gram stain results. If Gram stain is not performed or does not establish a diagnosis, antibiotics are selected on the basis of probabilities according to patient age, epidemiology, host risk factors, and severity of illness.

A severe atypical pneumonia, Severe acute respiratory syndrome (SARS), is a condition of unknown etiology that has been described very recently in patients in Asia, North America and Europe.

The majority of patients identified as having SARS have been adults aged 25-70 years who were previously healthy. Few suspected cases of SARS have been reported among children aged <15 years.

The incubation period for SARS is typically 2-7 days; however, isolated reports have suggested an incubation period as long as 10 days. The illness begins generally with a prodrome of fever (>100.4° F. [>38.0° C.]). Fever often is high, sometimes is associated with chills and rigors, and might be accompanied by other symptoms, including headache, malaise, and myalgia. At the onset of illness, some persons have mild respiratory symptoms. Typically, rash and neurologic or gastrointestinal findings are absent, however, some patients have reported diarrhea during the febrile prodrome.

After 3-7 days, a lower respiratory phase begins with the onset of a dry, nonproductive cough or dyspnea, which might be accompanied by or progress to hypoxemia. In 10%-20% of cases, the respiratory illness is severe enough to require intubation and mechanical ventilation. The case-fatality rate among persons with illness meeting the current WHO case definition of SARS is approximately 3%.

Chest radiographs might be normal during the febrile prodrome and throughout the course of illness. However, in a substantial proportion of patients, the respiratory phase is characterized by early focal interstitial infiltrates progressing to more generalized, patchy, interstitial infiltrates. Some chest radiographs from patients in the late stages of SARS also have shown areas of consolidation.

Early in the course of disease, the absolute lymphocyte count is often decreased. Overall white blood cell counts have generally been normal or decreased. At the peak of the respiratory illness, approximately 50% of patients have leukopenla and thrombocytopenia or low-normal platelet counts (50,000-150,000/μL). Early in the respiratory phase, elevated creatine phosphokinase levels (as high as 3,000 IU/L) and hepatic transaminases (two to six times the upper limits of normal) have been noted. In the majority of patients, renal function has remained normal.

The severity of illness might be highly variable, ranging from mild illness to death. Although a few close contacts of patients with SARS have developed a similar illness, the majority have remained well. Some dose contacts have reported a mild, febrile illness without respiratory signs or symptoms, suggesting the illness might not always progress to the respiratory phase.

The primary way that SARS appears to spread is by dose person-to-person contact Most cases of SARS have involved people who cared for or lived with someone with SARS, or had direct contact with infectious material (for example, respiratory secretions) from a person who has SARS. Potential ways in which SARS can be spread include touching the skin of other people or objects that are contaminated with infectious droplets and then touching your eye(s), nose, or mouth. This can happen when someone who is sick with SARS coughs or sneezes droplets onto themselves, other people, or nearby surfaces. It also is possible that SARS can be spread more broadly through the air or by other ways that are currently not known.

Information to date suggests that people are most likely to be infectious when they have symptoms, such as fever or cough. However, it is not known how long before or after their symptoms begin that patients with SARS might be able to transmit the disease to others.

Scientists at the Centers for Diseases Control and Prevention (CDC) and other laboratories have detected a previously unrecognized coronavirus in patients with SARS.

Currently, only preliminary data is available on the causative agent of this condition. A new coronavirus is the leading hypothesis for the cause of SARS (Ksiazek et al., A novel coronavirus associated with severe acute respiratory syndrome. The New England Journal of Medicine. WWW.neim.org 16 Apr. 2003). However, other viruses are still under investigation as potential causes.

Coronaviruses are a group of viruses that have a halo or crown-like (corona) appearance when viewed under a microscope. These viruses are a common cause of mild to moderate upper-respiratory illness in humans and are associated with respiratory, gastrointestinal, liver and neurologic disease in animals. Coronaviruses can survive in the environment for as long as three hours.

CDC scientists isolated a virus from the tissues of two SARS patients an d then used several laboratory methods to characterize it. Examination by electron microscopy revealed that the virus has the distinctive shape and appearance of coronaviruses, and genetic analysis suggests that this new virus does belong to the family of coronaviruses but differs from previously identified family members. Tests of serum specimens from people with SARS showed that they appeared to have been recently infected with this virus. Other tests demonstrated that this previously unrecognized coronavirus was present in a variety of clinical specimens (including specimens obtained by nose and throat swab) from other SARS patients with direct or indirect links to the outbreak. These results and other findings reported from laboratories participating in the World Health Organization (WHO) network provide growing evidence in support of the hypothesis that this new coronavirus is the cause of SARS. Additional studies of the link between this coronavirus and SARS are under way.

Coronaviruses have occasionally been linked to pneumonia in humans, especially people with weakened immune systems. The viruses also can cause severe disease in animals, including cats, dogs, pigs, mice, and birds.

Researchers from several laboratories participating in the WHO network have reported the identification of a paramyxovirus in clinical specimens from SARS patients. These laboratories are still investigating the possibility that a paramyxovirus is a cause of SARS.

At present the most efficacious treatment regimen, if any, is unknown. In several locations, therapy has included antivirals such as oseitamivir or ribavirin. Steroids also have been given orally or intravenously to patients in combination with ribavirin and other antimicrobials. In the absence of controlled clinical trials, however, the efficacy of these regimens remains unknown. Early information from laboratory experiments suggests that ribavirin does not inhibit virus growth or cell-to-cell spread of one isolate of the new coronavirus that was tested. Additional laboratory testing of ribavirin and other antiviral drugs is being done to see if an effective treatment can be found.

Interferons are cytokines, i.e. soluble proteins that transmit messages between cells and play an essential role in the immune system by helping to destroy microorganisms that cause infection and repairing any resulting damage. Interferons are naturally secreted by infected cells and were first identified in 1957. Their name is derived from the fact that they interfere with viral replication and production.

Interferons exhibit both antiviral and antiproliferative activity. On the basis of biochemical and immunological properties, the naturally-occurring human interferons are grouped into three major classes: interferon-alpha (leukocyte), interferon-beta (fibroblast) and interferon-gamma (immune). Alpha-interferon is currently approved in the United States and other countries for the treatment of hairy cell leukemia, venereal warts, Kaposi's Sarcoma (a cancer commonly afflicting patients suffering from Acquired Immune Deficiency Syndrome (AIDS)), and chronic non-A, non-B hepatitis.

Further, interferons (IFNs) are glycoproteins produced by the body in response to a viral infection. They inhibit the multiplication of viruses in protected cells. Consisting of a lower molecular weight protein, IFNs are remarkably non-specific in their action, i.e. IFN induced by one virus is effective against a broad range of other viruses. They are however species-specific, i.e. IFN produced by one species will only stimulate antiviral activity in cells of the same or a closely related species. IFNs were the first group of cytokines to be exploited for their potential anti-tumor and antiviral activities.

The three major IFNs are referred to as IFN-α, IFN-β and IFN-γ. Such main kinds of IFNs were initially classified according to their cells of origin (leukocyte, fibroblast or T cell). However, it became clear that several types might be produced by one cell. Hence leukocyte IFN is now caged IFN-α, fioroblast IFN is IFN-β and T cell IFN is IFN-γ. There is also a fourth type of IFN, lymphoblastoid IFN, produced in the “Namalwa” cell line (derived from Burkitt's lymphoma), which seems to produce a mixture of both leukocyte and fibroblast IFN.

The interferon unit or international unit for interferon (U or IU, for international unit) has been reported as a measure of IFN activity defined as the amount necessary to protect 50% of the cells against viral damage. The assay that may be used to measure bioactivity is the cytopathic effect inhibition assay as described (Rubinstein, et al. 1981; Familletti, P. C., et al., 1981). In this antiviral assay for interferon about 1 unit/ml of interferon is the quantity necessary to produce a cytopathic effect of 50%. The units are determined with respect to the international reference standard for Hu-IFN-beta provided by the National Institutes of Health (Pestka, S. 1986).

Every class of IFN contains several distinct types. IFN-β and IFN-γ are each the product of a single gene.

The proteins classified as IFNs-α are the most diverse group, containing about 15 types. There is a duster of IFN-α genes on chromosome 9, containing at least 23 members, of which 15 are active and transcribed. Mature IFNs-α are not glycosylated.

IFNs-α and IFN-β are all the same length (165 or 166 amino acids) with similar biological activities. IFNs-γ are 146 amino adds in length, and resemble the α and β classes less closely. Only IFNs-γ can activate macrophages or induce the maturation of killer T cells. These new types of therapeutic agents can are sometimes called biologic response modifiers (BRMs), because they have an effect on the response of the organism to the tumor, affecting recognition via immunomodulation.

Human fibroblast interferon (IFN-β) has antiviral activity and can also stimulate natural killer cells against neoplastic cells. It is a polypeptide of about 20,000 Da induced by viruses and double-stranded RNAs. From the nucleotide sequence of the gene for fibroblast interferon, cloned by recombinant DNA technology, (Derynk et al. 1980) deduced the complete amino add sequence of the protein. It is 166 amino add long.

Shepard et al. (1981) described a mutation at base 842 (Cys→Tyr at position 141) that abolished its anti-viral activity, and a variant done with a deletion of nucleotides 1119-1121.

Mark et al. (1984) inserted an artificial mutation by replacing base 469 (T) with (A) causing an amino add switch from Cys→Ser at position 17. The resulting IFN-β was reported to be as active as the ‘native’ IFN-β and stable during long-term storage (−70° C.).

Rebif® (recombinant human interferon-β), the latest development in interferon therapy for multiple sclerosis (MS), is interferon(IFN)-beta 1a, produced from mammalian cell lines.

The treatment of SRS with interferons alone or in combination with other anti-viral agents has not yet been reported in the literature.

### DESCRIPTION OF THE INVENTION

The main object of the present invention is the use of an interferon (IFN) alone or in combination with an antiviral agent for the manufacture of a medicament useful for treatment and/or prevention of Severe Acute Respiratory Syndrome (SARS).

The antiviral effects of interferons against two clinical isolates of the SARS-CoV (severe acute respiratory syndrome-associated coronavirus) have also been shown by some scientists at the University of Frankfurt (see J. Cintal et al., The Lancet, 362, 293-294, 2003). In this paper the scientists show that interferons inhibit SARS-CoV replication in vitro. In particular, they assessed the antiviral potential of recombinant interferons (IFN-alpha, IFN-beta and IFN-gamma) against two clinical isolates of SARS-CoV-FFM-1, from Frankfurt patients, and Hong Kong—replicated in Vero and Caco2 calls.

Interferon-beta was most potent, showing prophylactic protection and antiviral potential after injection in both isolates. Moreover the scientistst also tested the relevance of inhibition of virus replication for suppression of virus-induced cytopathogenic effects in cultures treated with interferon-beta 24 hours before and immediately after virus infection. Interferon-beta showed a dose-dependent inhibition of the production of infection virus in culture.

The term “treatment” within the context of this invention refers to any beneficial effect on progression of disease, including attenuation, reduction, decrease or diminishing of the pathological development after onset of disease.

An “interferon” or “IFN”, as used herein, is intended to include any molecule defined as such in the literature, comprising for example any types of IFNs mentioned in the above section “Background of the invention”. In particular, IFN-α, IFN-β and IFN-γ are included in the above definition. IFN-β is the preferred IFN according to the present invention. IFN-β suitable in accordance with the present invention is commercially available e.g. as Rebif® (Serono), Avonex® (Biogen) or Betaferon® (Schering). The use of interferons of human origin is also preferred in accordance with the present invention. The term interferon, as used herein, is intended to encompass salts, functional derivatives, variants, muteins, fused proteins, analogs and active fragments thereof.

The term “interferon-beta (IFN-β)”, as used herein, is intended to include fibroblast interferon in particular of human origin, as obtained by isolation from biological fluids or as obtained by DNA recombinant techniques from prokaryotic or eukaryotic host cells, as well as its salts, functional derivatives, variants, analogs and active fragments.

As used herein the term “muteins” refers to analogs of IFN in which one or more of the amino add residues of a natural IFN are replaced by different amino acid residues, or are deleted, or one or more amino add residues are added to the natural sequence of IFN, without changing considerably the activity of the resulting products as compared to the wild type IFN. These muteins are prepared by known synthesis and/or by site-directed mutagenesis techniques, or any other known technique suitable therefore. Preferred muteins include e.g. the ones described by Shepard et al. (1981) or Market al. (1984).

Any such mutein preferably has a sequence of amino adds sufficiently duplicative of that of IFN, such as to have substantially similar or even better activity to an IFN. The biological function of interferon is well known to the person skilled in the art, and biological standards are established and available e.g. from the National Institute for Biological Standards and Control (http://immunology.org/links/NIBSC).

Bioassays for the determination of IFN activity have been described. An IFN assay may for example be carried out as described by Rubinstein et al., 1981. Thus, it can be determined whether any given mutein has substantially a similar, or even a better, activity than IFN by means of routine experimentation.

Muteins of IFN, which can be used in accordance with the present invention, or nucleic add coding therefore, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino add substitutions of polypeptides or proteins of the invention, may include synonymous amino adds within a group, which have sufficiently similar physicochemical properties that substitution between members of the group will preserve the biological function of the molecule. It is dear that insertions and deletions of amino adds may also be made in the above-defined sequences without altering their function, particularly if the insertions or deletions only involve a few amino adds, e.g., under thirty, and preferably under ten, and do not remove or displace amino adds which are critical to a functional conformation, e.g., cysteine residues. Proteins and muteins produced by such deletions and/or insertions come within the purview of the present invention.

Preferably, the synonymous amino add groups are those defined in Table I. More preferably, the synonymous amino add groups are those defined in Table II: and most preferably the synonymous amino add groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met Tyr, Phe, Ile, Leu, Val |
| Gly | Ala, Thr, Pro, Ser, Gly |
| Ile | Met Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino add substitutions in proteins which can be used for obtaining muteins of IFN, for use in the present invention include any known method steps, such as presented in U.S. Pat. Nos. 4,959,314, 4,588,585 and 4,737,462, to Mark et al; U.S. Pat. No. 5,116,943 to Koths et al., U.S. Pat. No. 4,965,195to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al). Specific muteins of IFN-beta have been described, for example by Mark et al., 1984.

The term “fused protein” refers to a polypeptide comprising an IFN, or a mutein thereof, fused to another protein, which e.g., has an extended residence time in body fluids. An IFN may thus be fused to another protein, polypeptide or the like, e.g., an immunoglobulin or a fragment thereof.

“Functional derivatives” as used herein cover derivatives of IFN, and their muteins and fused proteins, which may be prepared from the functional groups which occur as side chains on the residues or the N— or C-terminal groups, by means known in the art, and are included in the invention as long as they remain pharmaceutically acceptable, i.e. they do not destroy the activity of the protein which is substantially similar to the activity IFN, and do not confer toxic properties on compositions containing it. These derivatives may, for example, include polyethylene glycol side-chains, which may mask antigenic sites and extend the residence of IFN in body fluids. Other derivatives include aliphatic esters of the carboxyl groups, amides of the carboxyl groups by reaction with ammonia or with primary or secondary amines, N-acyl derivatives of free amino groups of the amino add residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of IFN, or muteins and fused proteins, the present invention covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has no significantly reduced activity as compared to the corresponding IFN.

The term “salts” herein refers to both salts of carboxyl groups and to add addition salts of amino groups of the proteins described above or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral adds, such as, for example, hydrochloric add or sulfuric add, and salts with organic adds, such as, for example, acetic acid or oxalic add. Of course, any such salts must retain the biological activity of the proteins (IFN) relevant to the present invention, i.e., the ability to bind to the corresponding receptor and initiate receptor signaling.

In accordance with the present invention, antiviral can be used in combination with an interferon to potentiate its beneficial effects. According to the present invention, the use of Ribavirin (1-⊖-D-ribofuranosyl)-1H -1,2,4-Triazole-3-carboxamide), as antiviral is especially preferred.

In accordance with the present invention, the use of recombinant human IFN-beta and the compounds of the invention is further particularly preferred.

A special kind of interferon variant has been described recently. The so-called “consensus interferons” are non-naturally occurring variants of IFN (U.S. Pat. No. 6,013,253). According to a preferred embodiment of the invention, the compounds of the invention are used in combination with a consensus interferon.

As used herein, human interferon consensus (IFN-con) shall mean a non-naturally-occurring polypeptide, which predominantly includes those amino acid residues that are common to a subset of IFN-alpha's representative of the majority of the naturally-occurring human leukocyte interferon subtype sequences and which includes, at one or more of those positions where there is no amino add common to all subtypes, an amino add which predominantly occurs at that position and in no event includes any amino acid residue which is not existent in that position in at least one naturally-occurring subtype. IFN-con encompasses but is not limited to the amino acid sequences designated IFN-con1, IFN-con2 and IFN-con3 which are disclosed in U.S. Pat. Nos. 4,695,623, 4,897,471 and 5,541,293. DNA sequences encoding IFN-con may be produced as described in the above-mentioned patents, or by other standard methods.

In a further preferred embodiment, the fused protein comprises an 1 g fusion. The fusion may be direct, or via a short linker peptide which can be as short as 1 to 3 amino add residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of IFN and the immunoglobulin sequence. The resulting fusion protein may have improved properties, such as an extended residence time in body fluids (half-life), increased specific activity, increased expression level, or the purification of the fusion protein is facilitated.

In a further preferred embodiment, IFN is fused to the constant region of an 19 molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂, IgGₑ or IgG₄, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino add residues. Preferably, the moiety is a polyethylene (PEG) moiety. PEGylation may be carried out by known methods, such as the ones described in WO99/55377, for example.

The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired.

Standard dosages of human IFN-beta range from 80 000 IU/kg and 200 000 IU/kg per day or 6 MIU (million international units) and 12 MIU per person per day or 22 to 44 μg (microgram) per person. In accordance with the present invention, IFN may preferably be administered at a dosage of about 1to 50 μg, more preferably of about 10 to 30 μg or about 10 to 20 μg per person per day.

The administration of active ingredients in accordance with the present invention may be by intravenous, intramuscular or subcutaneous route. The preferred route of administration for IFN is the subcutaneous route.

IFN may also be administered daily or every other day, of less frequent Preferably, IFN is administered one, twice or three times per week.

The preferred route of administration is subcutaneous administration, administered e.g. three times a week. A further preferred route of administration is the intramuscular administration, which may e.g. be applied once a week.

Preferably 22 to 44 μg or 6 MIU to 12 MIU of IFN-beta is administered three times a week by subcutaneous injection.

IFN-beta may be administered subcutaneously, at a dosage of 25 to 30 μg or 8 MIU to 9.6 MIU, every other day.

30 μg or 9.6 MIU IFN-beta may further be administered intramuscularly once a week.

In a preferred embodiment Ribavirin is administered in combination with IFN-beta and it is administered at a dosage of about 100 to 2000 mg per person per day, preferably of about 400 to 1200 mg per person per day, more preferably about 800 to 1000 mg per person per day, or about 1000 to 1200 mg per person per day. For patients weighing less than 65 kg the usual dose is 800 mg per day, for patients weighing 65 to 85 kg the usual dose is 1000 mg per day and for patients weighing more than 85 kg the usual dose is 1200 mg per day. The actual dosage employed may be varied depending upon the requirements of the patient and the severity of the condition being treated. Determination of the proper dosage regimen for a particular situation is within the skill of the art For convenience, the total daily dosage may be divided and administered in portions during the day as required.

In a preferred embodiment, Ribavirin is administered orally.

Ribavirin may be administered by injection or, preferably, orally. Depending on the mode of administration, the compound can be formulated with the appropriate diluents and carriers to form ointments, creams, foams, and solutions having from about 0.01% to about 15% by weight, preferably from about 1% to about 10% by weight of the compound. For injection, Ribavirin is in the form of a solution or suspension, dissolved or suspended in physiologically compatible solution from about 10 mg/ml to about 1500 mg/ml. Injection may be intravenous, intermuscular, intracerebral, subcutaneous, or intraperitoneal.

For oral administration, Ribavirin may be in capsule, tablet, oral suspension, or syrup form. The tablet or capsules may contain from about 10 to 500 mg of Ribavirin. Preferably they may contain about 300 mg of Ribavirin. The capsules may be the usual gelatin capsules and may contain, in addition to the Ribavirin in the quantity indicated above, a small quantity, for example less than 5% by weight, magnesium stearate or other excipient. Tablets may contain the foregoing amount of the compound and a binder, which may be a gelatin solution, a starch paste in water, polyvinyl pyrilidone, polyvinyl alcohol in water, etc. with a typical sugar coating.

The compounds of the invention and IFN may be formulated in a pharmaceutical composition.

The term “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The subcutaneous route is preferred in accordance with the present invention.

Another possibility of carrying out the present invention is to activate endogenously the genes for IFN. In this case, a vector for including and/or enhancing the endogenous production of IFN in a cell normally silent for expression of IFN, or which expresses amounts of IFN which are not sufficient, are is used for treatment of SARS. The vector may comprise regulatory sequences functional in the cells desired to express IFN. Such regulatory sequences may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

The invention further relates to the use of a cell that has been genetically modified to produce IFN in the manufacture of a medicament for the treatment and/or prevention of SARS.

For parenteral (e.g. intravenous, subcutaneous, intramuscular) administration, IFN can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

According to the invention, the compounds of the invention and IFN can be administered prophylactically or therapeutically to an individual prior to, simultaneously or sequentially with other therapeutic regimens or agents (e.g. multiple drug regimens), in a therapeutically effective amount Active agents that are administered simultaneously with other therapeutic agents can be administered in the same or different compositions.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning of a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein. In combination with the knowledge of one of ordinary skill in the art.

### EXAMPLES

Clinical Trials

Clinical trials with 2 different doses of IFN-beta are carried out. The aim is to measure the clinical outcome of SARS CoV-infected patients. The clinical outcome will be to quantify the SARS-CoV viral titers in the nasopharyngeal aspirates and PBMC of the patients and to examine the immunological parameters that are predictive of the outcome

Design of the Clinical Trial

The first clinical trial is designed for children. The reasons for conducting the trial in children are the following. First of all the illness is shown to be less severe in children, for which no deaths have been registered until now. This would minimize the risk for treating patients who could be very sick and cannot tolerate initial doses of the drug. Moreover, the experience gained from the pediatric trial can be safely applied to adult patients.

A randomized control trial is performed to recruit patients younger than 18 years of age. The patients are selected on the basis of their clinical status and pulmonary radiographs by criteria as defined by World Health Organization. The patients are divided into 3 groups, consisting of 10 patients each: 1) control without IFN-beta, 2) IFN-beta at low dose (1 million units/m²/day), and 3) IFN-beta at medium dose (3 millionunits/m²/day). The patients are treated for 1 to 4 weeks depending on their clinical course. At the end of the treatment they are assessed for their clinical outcome, viral load, and immune responses.

Measure the Clinical Outcome of Co V-SARS-Infected Patients

The following patient data are collected for analysis during the course of the trial: fever*, chill or rigors, coughs*, dyspnea or respiratory distress*, myalgia, malaise, lethargy or irritability, poor feeding, rhinorrhea, sore throat, anorexia, diarrhea or vomiting, dizziness or neurological complaints, and rash. (*refers to prominent symptoms in SARS patients). Clinical outcome measurement is based on the hospital course, respiratory status of the patients (dyspnea or cyanosis), arterial blood gas results, the need for ventilatory support, and changes in pulmonary radiographs.

Determination SARS-CoV Viral Titers in the Nasopharyngeal Aspirates and Stool of the Patients.

Serial nasopharyngeal aspirates and stool samples are collected from the infected patents over a period of 3 weeks: before therapy, day 3, 6, 9, 12, 15, and 21. The samples will be cultured for SARS-CoV by using FRhK-4 cells. Indirect immunofluorescence assays are performed to characterize the infected cells. The cells will be examined by light microscopy for cytopathic effects and for the determination of viral titers per ml. Additionally, total RNA will be extracted from the samples for reverse transcription and subsequent Quantitative-PCR assays to identify the SARS-CoV using specific oligonucleotide primers.

Serum samples are collected for assaying SARS-CoV antibodies

Immunological Parameters Predictive of the Treatment Outcome

The expression of IFN-stimulated genes, which will be indicative of the effects of the exogenous IFN in vivo is measured. The IFN stimulated genes to be measured include 2-5 synthetase, PKR and Mx. These are well-established markers of IFN activity in the cells. Additionally, typical responders on terms of better clinical outcome and lower viral load after treatment) and non-responders (with poor clinical outcome and few or no changes in viral load after IFN-beta treatment) are selected.

When indicated, the gene expression profile of the patients' peripheral blood mononuclear cells is investigated by microarray systems (e.g. Affimetrix) and proteomics studies are carried out These results may be useful for identifying markers of therapeutic response.

### REFERENCES

1. Study Group. The Lancet 1998; 352, 1498-1504.
2. J. Cintal et al., The Lancet, 362, 293-294, 2003
3. Clegg and Bryant, Exp. Opin. Parmacother 2001; 2(4): 623-639.
4. Derynk R. et al., Nature 1980; 285, 542-547.
5. Familletti, P. C., Rubinstein, S., and Pestka, S. 1981 “A Convenient and Rapid Cytopathic Effect Inhibition Assay for Interferon,” in Methods in Enzymology, Vol. 78 (S. Pestka, ed.), Academic Press, New York, 387-394;
6. Hultgren C, Milich D R, Weiland O, Sallberg M. (1998). The antiviral compound ribavirin modulates the T helper (Th) 1/Th2 subset balance in hepatitis B and C virus-specific immune responses. J Gen Virol 1998: 79:2381-2391.
7. McCormick J B, King I J, Webb P A, Scribner C L, Craven R B, Johnson K M, Elliott L H, Belmont-Williams R. Lassa fever. Effective therapy with ribavirin. N Engl J Med. Jan. 2, 1986; 314(1):20-6.
8. Mark D. F. et al., Proc. Natl. Aced. Sci. U.S.A., 81 (18) 5662-5666 (1984).
9. Pestka, S. (1986) “Interferon Standards and General Abbreviations, in Methods In Enzymology (S. Pestka, ed.), Academic Press, New York 119, 14-23.
10. Rubinstein, S., Familletti, P. C., and Pestka. S. Convenient Assay for Interferons. J. Virol 1981; 37, 755-758.
11. Shepard H. M. et al., Nature 1981; 294, 563-565.
12. Tam R C, Pal B, Bard J, Lim C, Averett D R, Phan U T, Mllovanovic T. Ribavirin polarizes human T cell responses towards a Type 1 cytokine profile. J Hepatol. 1999; 30(3):376-82.
13. Togo Y. McCracken E. A., 1976. Double-blind clinical assessment of ribavirin (virazole) in the prevention of induced infection with type B influenza virus. J Infect Dis June 1976;133 Suppl: A109-13.

1-13. (canceled)

14. A method of treating Severe Acute Respiratory Syndrome (SARS) comprising the administration of a composition comprising an interferon (IFN) to an individual having SARS.

15. The method according to claim 14, wherein said composition comprises an IFN in combination with an antiviral agent.

16. The method according to claim 15, wherein said antiviral agent is Ribavirin.

17. The method according to claim 14, wherein said IFN is recombinant human IFN-beta.

18. The method according to claim 14, wherein said IFN is consensus interferon.

19. The method according to claim 15, wherein said IFN is recombinant human IFN-beta.

20. The method according to claim 15, wherein said IFN is consensus interferon.

21. The method according to claim 14, wherein said IFN is a fused protein comprising at least an immunoglobulin domain.

22. The method according to claim 15, wherein said IFN is a fused protein comprising at least an immunoglobulin domain.

23. The method according to claim 17, wherein said IFN is a fused protein comprising at least an immunoglobulin domain.

24. The method according to claim 18, wherein said IFN is a fused protein comprising at least an immunoglobulin domain.

25. The method according to claim 14, wherein said IFN is administered at a dosage of about 1 to 50 μg per person per day, or about 10 to 30 μg per person per day or about 10 to 20 μg per person per day.

26. The method according to claim 14, wherein said IFN is administered daily or every other day.

27. The method according to claim 14, wherein said IFN is administered twice or three times per week.

28. The method according to claim 14, wherein said IFN is administered subcutaneously.

29. The method according to claim 14, wherein said IFN is administered intramuscularly.

30. The method according to claim 15, wherein the antiviral agent is administered at a dosage of about 100 to 2000 mg per person per day, or about 400 to 1200 mg per person per day, or about 800 to 1000 mg per person per day, or about 1000 to 1200 mg per person per day.

31. The method according to claim 16, wherein Ribavirin is administered orally.

32. The method according to claim 14, wherein said method further comprises the administration of a composition comprising an antiviral agent to said individual.

33. The method according to claim 32, wherein said composition is administered simultaneously, sequentially or separately from a composition comprising an IFN.

34. The method according to claim 32, wherein said antiviral agent is Ribavirin.

35. The method according to claim 34, wherein said antiviral agent is administered at a dosage of about 100 to 2000 mg per person per day, or about 400 to 1200 mg per person per day, or about 800 to 1000 mg per person per day, or about 1000 to 1200 mg per person per day.

36. The method according to claim 35, wherein said antiviral agent is administered orally.
